(19) United States
(12) Patent Application Publication
(10) Pub. No.: US 2008/0200656 A1
(43) Pub. Date: Aug. 21, 2008

(54) Use Of Sucrose To Suppress Mannitol-Induced Protein Aggregation

(75) Inventors: David C. Sek, North Andover, MA (US); Nicholas W. Warne, Andover, MA (US); Kin Ho, Andover, MA (US); Donna L. Luisi, North Andover, MA (US); Angela Kantor, Pepperell, MA (US)

Correspondence Address: CHOATE, HALL & STEWART LLP/WYETH, PATENT GROUP, TWO INTERNATIONAL PLACE, BOSTON, MA, 02110, US

(73) Assignee: WYETH, Madison, NJ (US)

(21) Appl. No.: 12/032,490

(22) Filed: Feb. 15, 2008

Related U.S. Application Data
(60) Provisional application No. 60/901,807, filed on Feb. 16, 2007.

(51) Int. Cl. C07K 17/02

(52) U.S. Cl. 530/390.5

(57) ABSTRACT

The present invention provides a method for suppressing mannitol-induced protein aggregation by providing sucrose to a liquid formulation containing a protein. The present invention also provides methods for storing and preparing a liquid formulation containing a protein and a combination of mannitol and sucrose such that the presence of sucrose suppresses mannitol-induced protein aggregation.

### REFERENCE TO RELATED APPLICATIONS

This application claims the benefit of U.S. Provisional Patent Application No. 60/901,807, filed on Feb. 16, 2007, which is hereby incorporated by reference in its entirety.

### FIELD OF THE INVENTION

The present invention relates to methods for suppressing mannitol-induced protein aggregations in liquid formulations.

### BACKGROUND OF THE INVENTION

Mannitol has been generally used in protein formulations for maintaining stability and isotonicity of the formulation. In the past, liquid nitrogen has been used to quickly freeze protein formulations for storage. However, nearly all approaches to large-scale uncontrolled freezing of liquid formulations suffer from negative effects of uncontrolled solidification and melting. Inadequate control of phase change has been shown to result in product losses due to aggregation, precipitation, oxidation and denaturation. Recent technologies have been introduced to control the freeze and thaw process of protein formulations. However, these technologies typically freeze and thaw at a much slower rate. As a result, in mannitol-containing protein formulations, the slow freeze-thaw process allows crystallization of mannitol which, in turn, induces protein aggregation.

In order to avoid mannitol-induced protein aggregation during slow freeze-thaw processes, existing methods require removing mannitol from protein formulations and adding it back during post-thaw operation. These methods are expensive and require additional processing time.

### SUMMARY OF THE INVENTION

The present invention provides an improved method for suppressing mannitol-induced protein aggregations in liquid formulations. In particular, the present invention uses sucrose to suppress mannitol-induced protein aggregations in liquid formulations during freeze-thaw and storage. As a result, the present invention eliminates the need for removing and adding mannitol during, for example, drug product storage and filling operation. Therefore, the present invention reduces costs and processing time associated with storage and preparation of protein formulations.

In one aspect, the present invention provides a method for suppressing mannitol-induced protein aggregation by providing sucrose to a liquid formulation containing a protein. In some embodiments, the method of the present invention is used to suppress mannitol-induced protein aggregation during freeze and/or thaw processes. In some embodiments, the protein concentration in the liquid formulation is essentially constant during freeze-thaw, in particular, on a weight/weight basis. In some embodiments, the method of the present invention is used to suppress mannitol-induced protein aggregation during storage, in particular, at a frozen state (e.g., storage at a temperature within the range of 0° C. to −80° C., for instance, 0° C., −10° C., −20° C., −30° C., −40° C., −50° C., −60° C., −70° C., or −80° C., preferably 0° C. to −40° C., more preferably, −5° C. to −30° C.).

In some embodiments, the liquid formulation contains a combination of sucrose and mannitol. In some embodiments, the mannitol is in a concentration in the liquid formulation of no greater than about approximately 1 M and the sucrose is in a concentration in the liquid formulation of no greater than about approximately 5 M. In other embodiments, the mannitol is in a concentration in the liquid formulation of no greater than about approximately 300 mM and the sucrose is in a concentration in the liquid formulation of no greater than about approximately 300 mM. In some embodiments, the combined concentration of mannitol and sucrose in the liquid formulation is approximately 300 mM. The concentrations expressed in this paragraph may apply before the freezing step and/or after the thawing step.

In some embodiments, the molar ratio between mannitol and sucrose in the liquid formulation is within the range of 1:10 to 10:1, for instance, approximately 1:10, 1:5, 1:4, 1:3, 1:2, 1:1, 2:1, 3:1, 4:1, 5:1, 6:1, 7:1, 8:1, 9:1, or 10:1 (mannitol:sucrose). In some embodiments, the molar ratio between mannitol and sucrose in the liquid formulation is no greater than approximately 3:1 (mannitol:sucrose). In some embodiments, the molar ratio between mannitol and sucrose in the liquid formulation is no greater than approximately 5:1 (mannitol:sucrose).

In another aspect, the present invention provides a method for storing a liquid formulation including gradually cooling the liquid formulation to a temperature lower than −0° C. The liquid formulation includes a protein and a combination of mannitol and sucrose such that the presence of sucrose suppresses mannitol-induced protein aggregation during cooling. In some embodiments, the protein concentration in the liquid formulation is constant on a weight/weight basis during cooling.

In some embodiments, the method of this aspect of the invention includes gradually cooling the liquid formulation to a temperature within the range of 0° C. to −80° C., for instance, a temperature at or below approximately −10° C., −20° C., −30° C., −40° C., −50° C., −60° C., −70° C., or −80° C., preferably 0° C. to −40° C., more preferably, −5° C. to −30° C.

In some embodiments, the method of this aspect of the invention includes gradually cooling the liquid formulation at a rate within the range of 0.6° C./minute to 0.1° C./minute, for instance, at a rate of approximately 0.6, 0.5, 0.4, 0.3, 0.2, or 0.1° C./minute.

In some embodiments, the liquid formulation contains a protein that is an antibody. In particular, the antibody is preferably a monoclonal antibody. In other embodiments, the liquid formulation contains a protein that is a pharmaceutical drug substance.

In some embodiments, the method for storing a liquid formulation of this aspect of the invention is a process intermediate.

In some embodiments, the method of this aspect of the invention further includes a step of maintaining the liquid formulation at the temperature lower than 0° C. for a period of time. In some embodiments, the period of time is within the range of 1 month to 12 months, for instance, about 1 month or longer, 2 months or longer, 3 months or longer, 4 months or longer, 5 months or longer, 6 months or longer, 7 months or longer, 8 months or longer, 9 months or longer, 10 months or longer, 11 months or longer, or 12 months or longer.

In yet another aspect, the present invention provides a method for preparing a liquid formulation including gradually warming the liquid formulation to a temperature higher than 0° C. The liquid formulation includes a protein and a combination of mannitol and sucrose such that the presence of sucrose suppresses mannitol-induced protein aggregation during warming.

In some embodiments, the method of the present invention includes gradually warming the liquid formulation to a temperature within the range of 10° C. to 50° C. or higher, in particular, a temperature higher than approximately 10° C., 20° C., 25° C., 30° C., 37° C., 40° C., 50° C. or higher, preferably to ambient temperature. In some embodiments, the present invention includes gradually warming the liquid formulation from a frozen state (e.g., at temperature ranges given above, for instance, 0° C., −10° C., −20° C., −30° C., −40° C., −50° C., −60° C., −70° C., or −80° C.).

In some embodiments, the method of the present invention includes gradually warming the liquid formulation at a rate within the range of 0.6° C./minute to 0.1° C./minute, for instance, at a rate of approximately 0.6, 0.5, 0.4, 0.3, 0.2, or 0.1° C./minute.

In some embodiments, the liquid formulation contains a protein that is an antibody. In particular, the antibody is preferably a monoclonal antibody. In other embodiments, the liquid formulation contains a protein that is a pharmaceutical drug substance.

In some embodiments, the method for preparing a liquid formulation of this aspect of the invention is a process intermediate.

The present invention further provides a composition containing a biologically effective amount of the protein in the liquid formulation prepared by the method of the invention as described in various embodiments above.

In a further aspect, the present invention provides a method for processing a liquid formulation including: (1) providing a liquid formulation comprising a protein and a combination of mannitol and sucrose; (2) gradually freezing the liquid formulation; (3) gradually thawing the liquid formulation from the frozen state; and wherein the presence of sucrose suppresses mannitol-induced protein aggregation. In some embodiments, the method of the present invention is used to suppress mannitol-induced protein aggregation during freeze-thaw. In some embodiments, the method of the present invention is used to suppress mannitol-induced protein aggregation during storage, in particular, at a frozen state (e.g., storage at temperature ranges given above, for instance, 0° C., −10° C., −20° C., −30° C., −40° C., −50° C., −60° C., −70° C., or −80° C.).

In some embodiments, the method of this aspect of the invention further includes a step of maintaining the liquid formulation at a frozen state for a period of time following step (2) above. The period of time enables the formulation to be stored. In some embodiments, the period of time is within the range of 1 month to 12 months or longer, for instance, about 1 month or longer, 2 months or longer, 3 months or longer, 4 months or longer, 5 months or longer, 6 months or longer, 7 months or longer, 8 months or longer, 9 months or longer, 10 months or longer, 11 months or longer, or 12 months or longer.

In some embodiments, the gradual freezing or thawing is at a rate within the range of 0.6 to 0.1° C./minute, preferably approximately 0.6, 0.5, 0.4, 0.3, 0.2, or 0.1° C./minute.

In some embodiments, the liquid formulation contains a protein that is an antibody. In particular, the antibody is a monoclonal antibody. In other embodiments, the liquid formulation contains a protein that is a pharmaceutical drug substance.

In some embodiments, the method for storing a liquid formulation of this aspect of the invention is a process intermediate.

The present invention further provides a composition containing a biologically effective amount of the protein stored in the liquid formulation by the method of the invention as described in various embodiments above.

The present invention as described above in various embodiments may be used to store and/or to prepare a liquid formulation containing a solubilized protein at any given concentration. For example, the liquid formulation may contain a protein in a concentration at or below about 10 mg/ml, 20 mg/ml, 25 mg/ml, 30 mg/ml, 35 mg/ml, 40 mg/ml, 45 mg/ml, 49 mg/ml, 75 mg/ml, 100 mg/ml, 125 mg/ml, 150 mg/ml, 175 mg/ml, 200 mg/ml. In other embodiments, the liquid formulation may contain a protein in a concentration at or greater than about 10 mg/ml, 20 mg/ml, 25 mg/ml, 30 mg/ml, 35 mg/ml, 40 mg/ml, 45 mg/ml, 49 mg/ml, 75 mg/ml, 100 mg/ml, 125 mg/ml, 150 mg/ml, 175 mg/ml, 200 mg/ml.

Typically, the liquid formulation according to the invention is an aqueous formulation.

Typically, the freezing or cooling step used in the present invention as described in various embodiments above is not accompanied by a simultaneous drying process, such as one used in a lyophilization process. In particular, the concentration of the protein, the mannitol or sucrose is essentially constant on a weight/weight basis during cooling or freezing step.

In this application, the use of “or” means “and/or” unless stated otherwise. As used in this disclosure, the term “comprise” and variations of the term, such as “comprising” and “comprises,” are not intended to exclude other additives, components, integers or steps. As used in this application, the terms “about” and “approximately” are used as equivalents. Both terms are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIG. 1 illustrates sample product temperature traces at exemplary process scales with a CryoPilot (CP) system.

FIG. 2 illustrates that mannitol induces antibody aggregation during freeze-thaw cycles.

FIG. 3 illustrates exemplary SEC-HPLC chromatograms of proteins frozen and thawed at controlled rates with varying ratios of mannitol and sucrose.

FIG. 4 illustrates the high molecular weight (HMW) species formation as a function of mannitol:sucrose ratio.

FIG. 5A depicts an exemplary experiment showing that sucrose suppresses the mannitol-induced protein aggregation during storage at −20° C.

FIG. 5B depicts an exemplary experiment showing that the formation of LMW species were comparable among the formulations with or without sucrose during storage at −20° C.

### DETAILED DESCRIPTION OF THE INVENTION

The present invention provides an improved method for suppressing mannitol-induced protein aggregations in liquid formulations. Specifically, the present invention uses sucrose to suppress mannitol-induced protein aggregations in liquid formulations during freeze-thaw and storage. The invention also provides methods for storing or preparing a liquid formulation containing a protein and a combination of mannitol and sucrose such that the presence of sucrose suppresses mannitol-induced protein aggregation.

Various aspects of the invention are described in further detail in the following subsections. The use of subsections is not meant to limit the invention. Each subsection may apply to any aspect of the invention.

### Protein Formulations

Proteins are relatively unstable in the aqueous state and undergo chemical and physical degradation resulting in a loss of biological activity during processing and storage. Freeze-thaw and lyophilisation are well-established methods for preserving proteins for storage. In order to preserve protein conformation, activity and stability, the protein formulations usually contain agents facilitating this, so-called lyoprotectants and cryoprotectants. Cryoprotectants are agents which provide stability to the protein from freezing-induced stresses; however, the term also includes agents that provide stability, e.g., to bulk drug formulations during storage from non-freezing-induced stresses. Lyoprotectants are agents that provide stability to the protein during water removal from the system during the drying process, presumably by maintaining the proper conformation of the protein through hydrogen bonding. Cryoprotectants can also have lyoprotectant effects. Examples of frequently used bulking agents include mannitol, glycine, lactose, etc. The agents also contribute to the tonicity of the formulations.

As used herein, “proteins” include any polypeptides. Exemplary proteins include, but are not limited to, antibodies, e.g., monoclonal antibodies, single chain antibodies, and other antibody variants; various growth hormones; any pharmaceutical drug substances. Proteins referred to in this application include any naturally-occurring, modified or synthesized polypeptides.

As used herein, “a protein formulation,” “a liquid formulation,” or grammatical equivalents include any liquid polypeptide-containing compositions. The liquid polypeptide-containing compositions may further contain “buffering agent” including those agents which maintain the solution pH in an acceptable range and may include bulking agents described above and may also include histidine, phosphate, citrate, tris, diethanolamine, and the like. If the liquid polypeptide-containing compositions are pharmaceutical compositions, the liquid formulation may further contain “excipients.” The term “excipients” includes pharmaceutical acceptable carriers as well as lyoprotectants and cryoprotectants that provide proper conformation of the protein during storage so that substantial retention of biological activity and protein stability is maintained.

### Mannitol Induces Protein Aggregation During Slow Freeze and Thaw

As discussed above, freeze and thaw is a well establish method for long-term storage or as an intermediate step. However, nearly all approaches to large-scale freezing of liquid formulations suffer from negative effects of uncontrolled solidification and melting. Approaches such as freezing in bags and bottles have been repeatedly shown to result in cryoconcentration and non-uniform temperature profiles within containers. Inadequate control of phase change has been shown to result in product losses due to aggregation, precipitation, oxidation and denaturation. By contrast, controlled freeze and thaw (also referred to as slow freeze and thaw) avoids product denaturation typical of uncontrolled methods and eliminates expensive and time-consuming cleaning. In addition, overall processes benefit from a well-controlled and predictable operation.

Controlled freezing (or slow freezing) typically includes gradually freezing or cooling a liquid formulation to a temperature suitable for storage at a predetermined rate. Typically, a temperature suitable for storage includes, but is not limited to, a temperature at or below about 0° C., −10° C., −20° C., −30° C., −40° C., −50° C., −60° C., −70° C., or −80° C. The gradual step down cooling can be at any suitable rate. For example, a step down cooling rate can be within the range of 0.6 to 0.1° C./minute, for instance, approximately 0.6, 0.5, 0.4, 0.3, 0.2, or 0.1° C./minute.

Similarly, controlled thawing (slow thawing) typically includes gradually thawing or warming a liquid formulation to a desired temperature at a predetermined rate. In particular, the liquid formulation is thawed or warmed from a frozen state. Typically, a desired temperature for thawing purposes includes, but is not limited to, a temperature at or above about 0° C., 10° C., 20° C., 30° C., 37° C., 40° C., 45° C., or 50° C. In some embodiments, the suitable temperature is 37° C. The gradual step warming can be at any suitable rate. For example, a gradual step warming rate can be within the range of 0.6 to 0.1° C./minute, for instance, approximately 0.6, 0.5, 0.4, 0.3, 0.2, or 0.1° C./minute.

Controlled freeze and/or thaw may be performed in a container, such as a tube, a bag, a bottle, or any other suitable containers. The containers may be disposable. Controlled freeze and/or thaw may also be performed in a large scale or small scale. For typical large scale production, a liquid formulation may be frozen in batches of about 1 L through 300 L, for example, 1 L, 3 L, 10 L, 20 L, 50 L, 100 L, 125 L, 250 L, or 300 L. For typical small scale system, a liquid formulation may be frozen in batches of about 1 ml to 500 ml, for example, 1 ml, 10 ml, 20 ml, 30 ml, 50 ml, 100 ml, 200 ml, 300 ml, 400 ml, or 500 ml.

However, in mannitol-containing liquid formulations, the slow freezing and/or thawing allows crystallization of mannitol, which in turn, induces protein aggregation. As used herein, “protein aggregation” is meant formation of high molecular weight (HMW) species including both insoluble species detectable by turbidity measurement and soluble species detectable by size-exclusion chromatography HPLC (SEC-HPLC), cation exchange-HPLC (CEX-HPLC), X-ray diffraction (XRD), modulated differential scanning calorimetry (mDSC) and other means known to one of skill in the art.

It is observed that there is a substantial increase in the percentage of HMW species in mannitol-containing formulations upon multiple freeze and thaw cycles (see the Examples section) or after long term storage, in particular, at a frozen state (e.g., at approximately 0° C., −10° C., −20° C., −30° C., −40° C., −50° C., −60° C., −70° C., or −80° C.). Increased amount of mannitol in the formulation also results in higher percentage of HMW species formation. Reduced processing volume appears to maintain the percentage of HMW species formed compared to large scale (e.g., 125 L).

An exothermal event is observed during cooling in mannitol-containing formulations. The observed enthalpy, which is due to the crystallization of mannitol as well as to the unfrozen water, increases as the processing scale increases (freeze and thaw rates decreases), or the mannitol level in the formulation increases. Crystallization event upon thawing in the mannitol-containing formulation is also observed. Without wishing to be bound by theory, the crystallization events in frozen solution suggest that the phase transition due to crystallization may induce the aggregation of protein upon freeze and thaw. Crystallization of mannitol increases with the mannitol level, which corresponds to higher % HMW formation. There was more mannitol crystallization observed in larger process scale simulation than that of the smaller scale, again correlated to greater rate of HMW formation. Decreasing mannitol in the formulation generally favors reducing HMW species formation in the liquid formulation during freeze and thaw.

### Use of Sucrose to Suppress Mannitol-Induced Protein Aggregation

In order to address the mannitol-induced protein aggregation and to provide liquid formulations that stabilize proteins during freeze-thaw or long-term storage, the present invention investigated whether and how the addition of a different cryoprotectant affects the mannitol-induced protein aggregation. Cryoprotectants is excipients that protect the product against the freeze and thaw of the solution. Typically, cryoprotectants include, but are not limited to, carbohydrates or polyols. As described in Example 2, the present invention discovered that providing sucrose to the liquid formulation suppresses or inhibits mannitol-induced protein aggregation during freeze-thaw. In addition, as described in Example 3, sucrose suppresses mannitol-induced protein aggregation during long term storage. A protein formulation containing sucrose, even at a low concentration, is stable after long term storage. As used herein, the terms “suppresses protein aggregation,” “inhibits protein aggregation,” or grammatical equivalents, denotes a reduction of the percentage of HMW species in a liquid formulation containing a combination of sucrose and mannitol as compared to the percentage of HMW species formed in a similar liquid formulation containing mannitol without sucrose. The terms “suppresses protein aggregation” or “inhibits protein aggregation” also include eliminating formation of HMW species.

Thus, the present invention provides a method for suppressing mannitol-induced protein aggregation by providing sucrose to a liquid formulation that contains a protein. Typically, the liquid formulation of the invention contains a combination of mannitol and sucrose. The sucrose and mannitol may be present in any concentration, only limited by their respective maximum solubilities. For example, the mannitol may be present in any concentration less than approximately 1 M and the sucrose may be present in any concentration less than approximately 5M. In some embodiments, the mannitol may be present in a concentration no greater than approximately 300 mM and the sucrose may be present in a concentration no greater than approximately 300 mM. In some embodiments, the combined concentration of mannitol and sucrose is approximately 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 600 mM, 700 mM, 800 mM, 900 mM, 1M or higher.

The mannitol and sucrose can be at any ratio in a liquid formulation. In some embodiments, the molar ratio between mannitol and sucrose in the liquid formulation is approximately 1:10, 1:5, 1:4, 1:3, 1:2, 1:1, 2:1, 3:1, 4:1, 5:1, 6:1, 7:1, 8:1, 9:1, or 10:1 (mannitol:sucrose). In some embodiments, the molar ratio between mannitol and sucrose in the liquid formulation is no greater than approximately 3:1 (mannitol:sucrose). In some embodiments, the molar ratio between mannitol and sucrose in the liquid formulation is no greater than approximately 5:1 (mannitol:sucrose).

As non-limiting examples, a suitable sucrose concentration in a liquid formulation can be within the range of 10 mM to 5M, for instance, 10 mM, 20 mM, 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM 1M, 1.5M, 2M, 2.5M, 3M, 3.5M, 4M, 4.5M, or 5M.

As non-limiting examples, a suitable mannitol concentration in a liquid formulation can be within the range of 10 mM to 1M, for instance, 10 mM, 20 mM, 30 mM, 40 mM, 50 mM, 60 mM, 70 mM, 80 mM, 90 mM, 100 mM, 125 mM, 150 mM, 175 mM, 200 mM, 225 mM, 250 mM, 275 mM, 300 mM, 325 mM, 350 mM, 375 mM, 400 mM, 425 mM, 450 mM, 475 mM, 500 mM, 600 mM, 700 mM, 800 mM, 900 mM or 1M.

The present invention discovered that sucrose may be used in a variety of liquid formulations to inhibit mannitol-induced protein aggregation during freezing, thawing, or storage. The liquid formulation may contain a solubilized protein at any given concentration. For example, the liquid formulation may contain a protein in a concentration at or below about 10 mg/ml, 20 mg/ml, 25 mg/ml, 30 mg/ml, 35 mg/ml, 40 mg/ml, 45 mg/ml, 49 mg/ml, 75 mg/ml, 100 mg/ml, 125 mg/ml, 150 mg/ml, 175 mg/ml, 200 mg/ml. In other embodiments, the liquid formulation may contain a protein in a concentration at or greater than about 10 mg/ml, 20 mg/ml, 25 mg/ml, 30 mg/ml, 35 mg/ml, 40 mg/ml, 45 mg/ml, 49 mg/ml, 75 mg/ml, 100 mg/ml, 125 mg/ml, 150 mg/ml, 175 mg/ml, 200 mg/ml.

Sucrose can be used in a liquid formulation containing any protein as described above or known in the art. For example, the protein may be an antibody. In particular, the antibody may be a monoclonal antibody, or single chain antibody, or other antibody variants. The protein may also be a growth hormone or a pharmaceutical drug substance. The protein may be a naturally-occurring, modified or synthesized polypeptide. The protein may be a small or large molecule. For example, the protein may have a molecular weight at or greater than approximately 25 kDa, 50 kDa, 75 kDa, 100 kDa, 125 kDa, 150 kDa, 175 kDa, 200 kDa, 225 kDa, 250 kDa, 275 kDa, or 300 kDa. The protein may be a monomer, a dimer, or a multimer.

Thus, by adding sucrose in a mannitol-containing liquid formulation, the present invention allows slow freezing and/or thawing of the liquid formulation without inducing significant protein aggregation. By adding sucrose in a mannitol-containing liquid formulation, the present invention also allows long-term storage of the liquid formulation at a frozen state without inducing protein aggregation. The present invention is particularly useful for storing drug product containing drug substance. For example, the present invention allows all the excipients in a drug product to be present during slow freezing and/or thawing process while keeping the drug substance stable and biologically active. Therefore, the present invention eliminates the need for removing mannitol from a drug formulation before storage and adding it back during the drug product filling operation. The present invention also prevents the need for having to concentrate drug substance up to high concentrations in order to be able to add mannitol during the drug product filling operation.

Thus, the liquid formulations containing a protein and a combination of mannitol and sucrose may be stored directly in that form for later use, stored at a frozen state (e.g., stored at a temperature within the range of 0° C. to −80° C., for instance, 0° C., −10° C., −20° C., −30° C., −40° C., −50° C., −60° C., −70° C., or −80° C.) as an intermediate step and thawed prior to use, or subsequently prepared in a dried form, such as a lyophilized, air-dried, or spray-dried form, for later reconstitution into a liquid form or other form prior to use. In addition, compositions containing biologically active amount of the protein can be prepared and stored directly in their liquid form in accordance with the present application to take full advantage of the convenience, ease of administration without reconstitution, and ability to supply the formulation in prefilled, ready-to-use syringes or as multidose preparations if the formulation is compatible with bacteriostatic agents. The present application also provides other forms of compositions containing biologically active amount of the protein in the liquid formulation stored and prepared as described above.

It should be understood that the above-described embodiments and the following examples are given by way of illustration, not limitation. The liquid formulation of the present invention is applicable to proteins in general. For example, the antibodies used in the liquid formulations described in the Examples section can be any antibodies. Various changes and modifications within the scope of the present invention will become apparent to those skilled in the art from the present description.

### EXAMPLES

### Example 1

### Mannitol Induces Protein Aggregation During Slow Freeze and Thaw

A formulation containing a monoclonal antibody (referred to as MAB-001 in this experiment) and 10 mM histidine, 10 mM methionine, 4% mannitol and 0.005% polysorbate-80, pH 6.0, was frozen and thawed multiple times using a CryoPilot (CP) system (Stedim Biosystems). Each freeze and thaw profile included step-down cooling to −55° C., and warming to 32° C. while the solution was mixed.

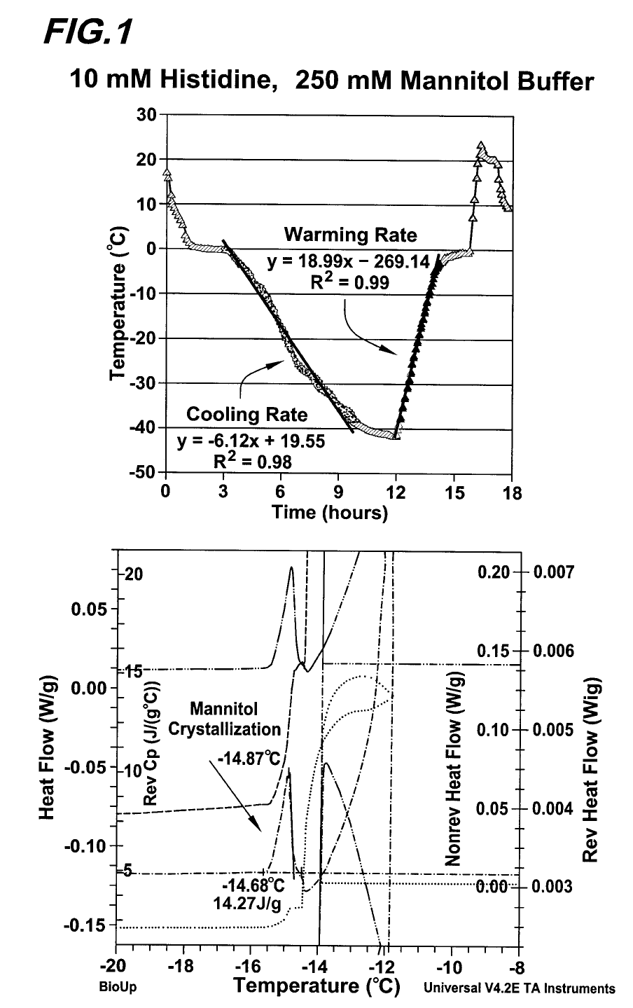

The CP simulates operation of a CryoVessel (Stedim Biosystems), the full scale production unit. The CP set point profiles for various process volumes had been developed prior to this work, to mimic behavior of the CryoVessel. FIG. 1 illustrates a sample of product temperature trace at each process scale with the CP system. Freezing (or thawing) rate was defined as the thermocouple reaching −42° C. from 0° C. (or 0° C. from −42° C.) divided by the time.

Thawed samples were analyzed primarily by SEC-HPLC and CEX-HPLC to evaluate the level of high molecular weight species (% HMW), and track the levels of acidic and basic species. Modulated differential scanning calorimetry (mDSC) and X-Ray Diffraction (XRD) were also used to assess crystallinity and polymorphs of mannitol in frozen solutions.

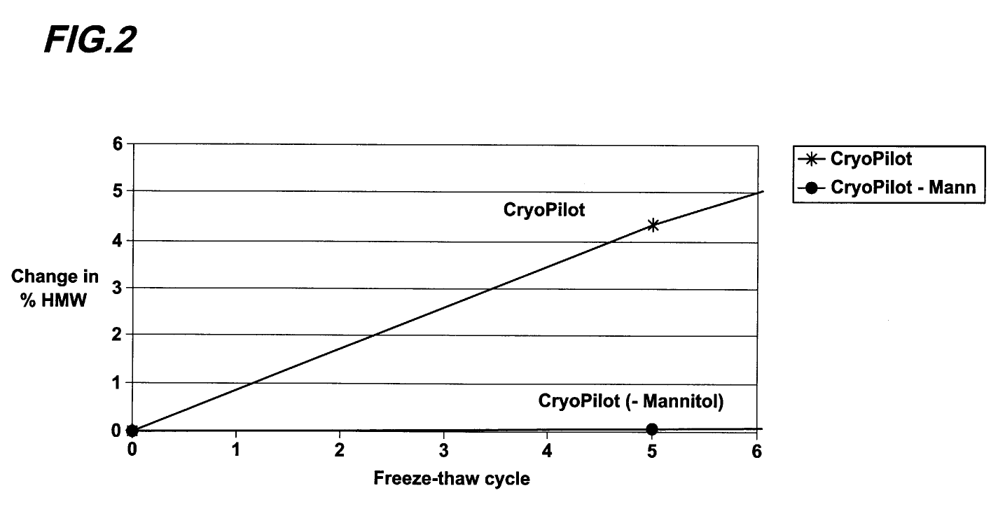

MAB-001 was found to aggregate in the presence of mannitol during slow freeze-thaw process described above. FIG. 2 illustrates formation of HMW species of MAB-001 during freeze-thaw cycles.

### Example 2

### Sucrose Suppresses Mannitol-Induced Protein Aggregation

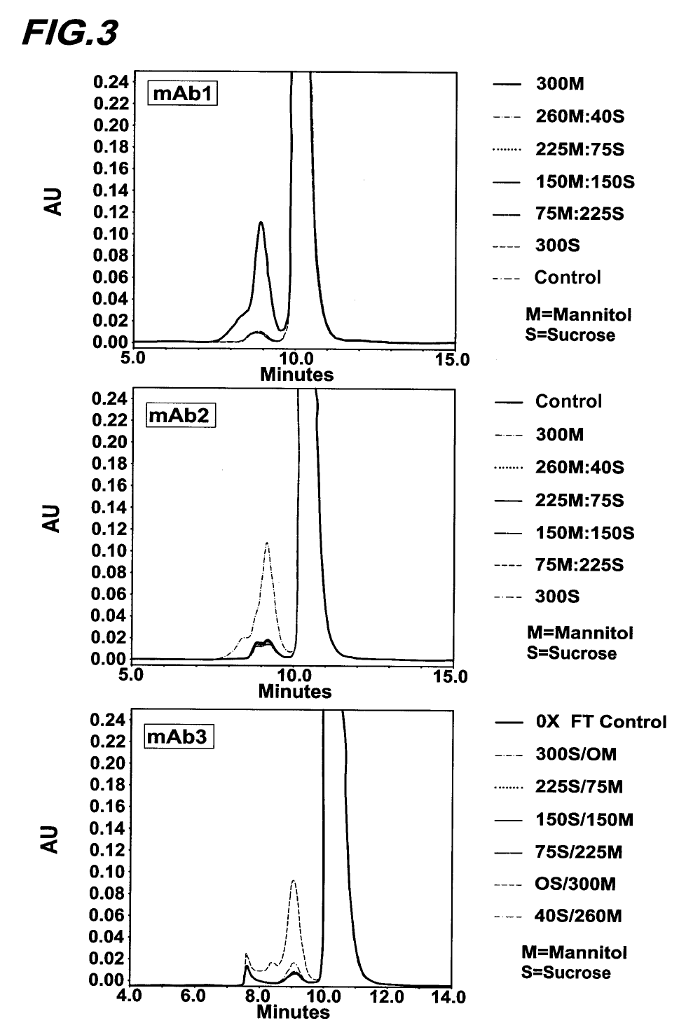

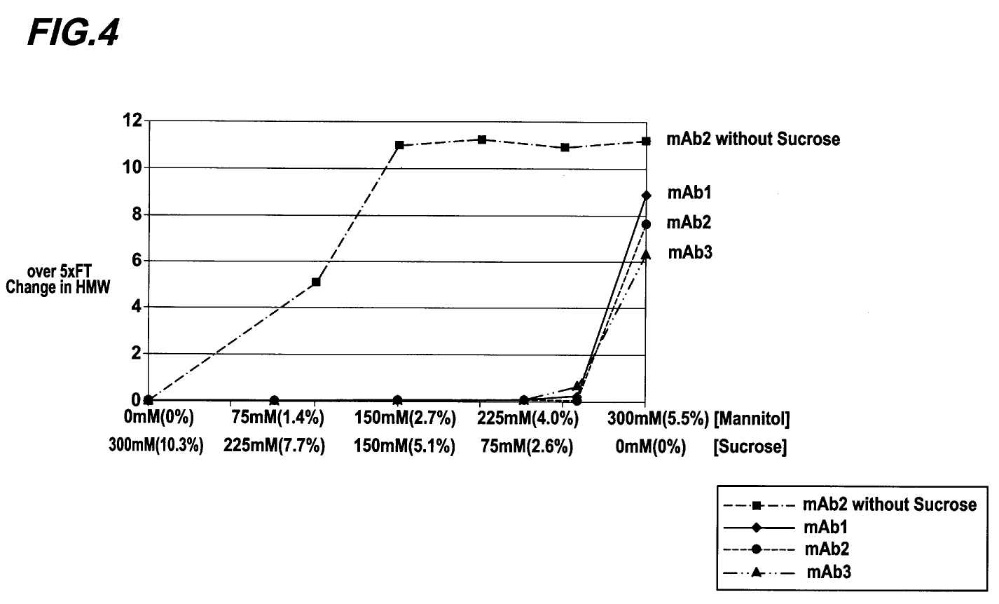

Three different monoclonal antibodies (referred to as mAb1, mAb2, and mAb3) were dialyzed into 10 mM histidine, 300 mM mannitol, pH 6.0, or 10 mM histidine, 300 mM sucrose, pH 6.0. The two formulations were combined to create varying ratios of mannitol to sucrose, such as, 300:0, 225:75, 150:150, 75:225, 40:260, 0:300 (mannitol:sucrose). Protein concentration in each formulation was normalized to 20 mg/ml. These formulations were then subject to five cycles of freeze-thaw as described above, and monitored for HMW species formation by SEC-HPLC. Exemplary SEC-HPLC chromatograms were shown in FIG. 3. Changes in formation of HMW species over the course of five cycles of freeze-thaw at controlled rates were plotted against the molar ratios of mannitol to sucrose in FIG. 4. As shown in FIG. 4, the presence of sucrose significantly suppresses mannitol-induced protein aggregations in liquid formulations during freeze-thaw. In particular, as shown in FIG. 4, the presence of sucrose in a molar ratio of 1:3 sucrose:mannitol is sufficient to eliminated aggregation in all three monoclonal antibody formulations.

### Example 3

### Sucrose Suppresses Mannitol-Induced Protein Aggregation During Long Term Frozen Storage

A monoclonal antibody was dialyzed into 10 mM histidine, 300 mM mannitol, pH 6.0, or mM histidine, 300 mM sucrose, pH 6.0. The two formulations were combined to create varying ratios of mannitol to sucrose, such as, 300 mM:0 mM, 200 mM:100 mM, 100 mM:200 mM, 50 mM:250 mM, 0 mM:300 mM (mannitol:sucrose). The control formulation contains 10 mM histidine without mannitol or sucrose. Protein concentration in each formulation was normalized to 20 mg/ml.

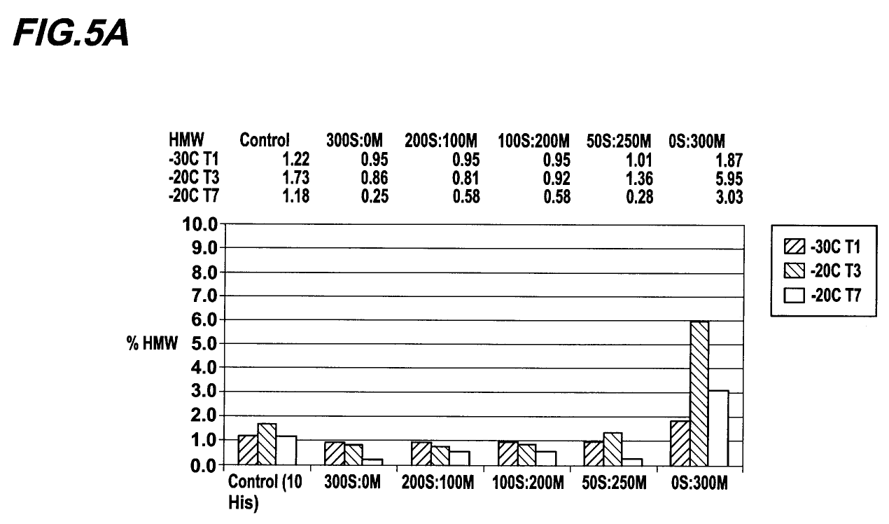

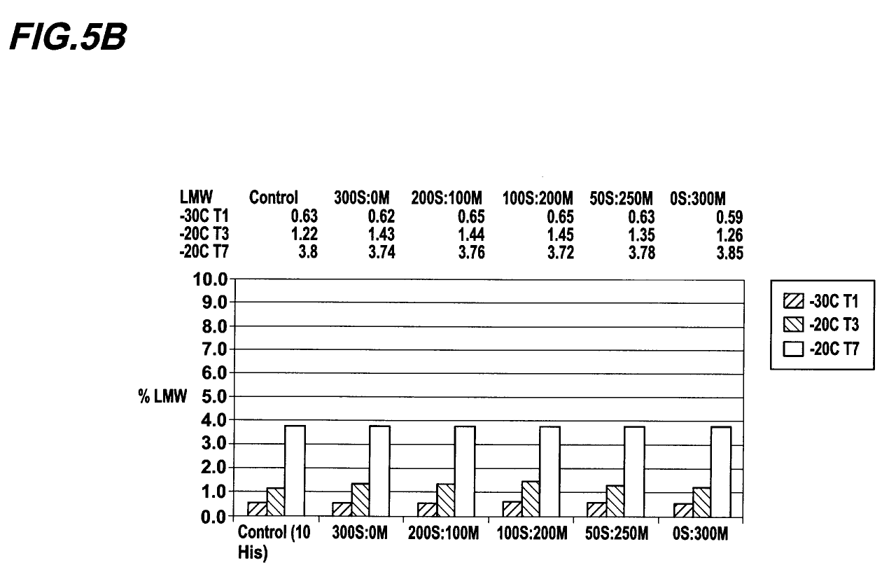

The effect of sucrose during long term frozen storage were evaluated as follows. The control and the formulations containing varying ratios of mannitol to sucrose as described above were first cooling down to −30° C., then stored at −20° C. Samples were taken after the formulations were frozen down to −30° C. (T1), then after 3 months (T3) and 7 months (T7) storage at −20° C. and assayed for HMW species formation by SEC-HPLC. As shown in FIG. 5A, the formation of HMW in the control formulation increased after storage for 3 months at −20° C., which indicates that, without a stabilizer, the protein aggregates during frozen storage (see, the control in FIG. 5A). The formulation containing 300 mM mannitol without sucrose showed a significantly larger increase in the formation of HMW than the control after 3 months storage at −20° C., which indicates that mannitol induces protein aggregation (see, the formulation containing 300 mM mannitol without sucrose in FIG. 5A). The formation of HMW in the formulations containing sucrose was greatly reduced during frozen storage (FIG. 5A). For example, as shown in FIG. 5A, 50 mM sucrose was sufficient to suppress mannitol-induced protein aggregation during long-term frozen storage, which indicates, that, sucrose, even at a low concentration, inhibits mannitol-induced protein aggregation. In addition, the formulations containing sucrose at a concentration of 50 mM, 100 mM, 200 mM, and 300 mM, respectively, were stable at −20° C. after long-term storage (FIG. 5A). As shown in FIG. 5B, the formation of LMW were comparable among the control and the formulations with or without sucrose after long-term frozen storage.

### EQUIVALENTS

The foregoing has been a description of certain non-limiting embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

### INCORPORATION BY REFERENCE

All publications and patent documents cited in this application are incorporated by reference in their entirety for all purposes to the same extent as if the contents of each individual publication or patent document were incorporated herein.

What is claimed is:

1. A method for suppressing mannitol-induced protein aggregation in a liquid formulation: during freeze-thaw, the method comprising providing sucrose to a liquid formulation comprising a protein, wherein the protein concentration in the liquid formulation is essentially constant on a weight/weight basis during freeze-thaw.

2. The method of claim 1, wherein the liquid formulation comprises a combination of sucrose and mannitol.

3. The method of claim 2, wherein the mannitol is in a concentration no greater than approximately 1 M and the sucrose is in a concentration no greater than approximately 5 M.

4. The method of claim 2, wherein the mannitol is in a concentration no greater than approximately 300 mM and the sucrose is in a concentration no greater than approximately 300 mM.

5. The method of claim 4, wherein the combined concentration of mannitol and sucrose is approximately 300 mM.

6. The method of claim 2, wherein the mannitol and sucrose are at a molar ratio no greater than approximately 3:1 (mannitol:sucrose).

7. The method of claim 2, wherein the mannitol and sucrose are at a molar ratio no greater than approximately 5:1 (mannitol:sucrose).

8. A method for storing a liquid formulation, the method comprising gradually cooling a liquid formulation to a temperature lower than −0° C., wherein the liquid formulation comprises a protein and a combination of mannitol and sucrose such that the presence of sucrose suppresses mannitol-induced protein aggregation during cooling, and wherein the protein concentration in the liquid formulation is essentially constant on a weight/weight basis during freeze-thaw.

9. The method of claim 8, wherein the mannitol is in a concentration no greater than approximately 1 M and the sucrose is in a concentration no greater than approximately 5 M.

10. The method of claim 9, wherein the mannitol is in a concentration no greater than approximately 300 mM and the sucrose is in a concentration no greater than approximately 300 mM.

11. The method of claim 10, wherein the combined concentration of mannitol and sucrose is approximately 300 mM.

12. The method of claim 8, wherein the mannitol and sucrose are at a molar ratio no greater than approximately 3:1 (mannitol:sucrose).

13. The method of claim 8, wherein the mannitol and sucrose are at a molar ratio no greater than approximately 5:1 (mannitol:sucrose).

14. The method of claim 8, wherein the temperature is lower than −10° C.

15. The method of claim 8, wherein the temperature is lower than −20° C.

16. The method of claim 8, wherein the temperature is lower than −50° C.

17. The method of claim 8, wherein the cooling is at a rate of approximately 0.5° C./minute.

18. The method of claim 8, wherein the cooling is at a rate of approximately 0.3° C./minute.

19. The method of claim 8, wherein the cooling is at a rate of approximately 0.1° C./minute.

20. The method of claim 8, wherein the protein is an antibody.

21. The method of claim 20, wherein the antibody is a monoclonal antibody.

22. The method of claim 8, wherein the protein is a pharmaceutical drug substance.

23. The method of claim 8, wherein the method is a process intermediate.

24. The method of claim 8, wherein the method further comprises a step of maintaining the liquid formulation at the temperature lower than 0° C. for a period of time.

25. The method of claim 24, wherein the period of time is 6 months or longer.

26. A method for preparing a liquid formulation, the method comprising gradually warming a liquid formulation to a temperature higher than 0° C., wherein the liquid formulation comprises a protein and a combination of mannitol and sucrose such that the presence of sucrose suppresses mannitol-induced protein aggregation during warming.

27. The method of claim 26, wherein the mannitol is in a concentration no greater than approximately 1 M and the sucrose is in a concentration no greater than approximately 5 M.

28. The method of claim 27, wherein the mannitol is in a concentration no greater than approximately 300 mM and the sucrose is in a concentration no greater than approximately 300 mM.

29. The method of claim 28, wherein the combined concentration of mannitol and sucrose is approximately 300 mM.

30. The method of claim 26, wherein the mannitol and sucrose are at a molar ratio no greater than approximately 3:1 (mannitol:sucrose).

31. The method of claim 26, wherein the mannitol and sucrose are at a molar ratio no greater than approximately 5:1 (mannitol:sucrose).

32. The method of claim 26, wherein the temperature is higher than 20° C.

33. The method of claim 26, wherein the temperature is higher than 30° C.

34. The method of claim 26, wherein the warming is at a rate of approximately 0.5° C./minute.

35. The method of claim 26, wherein the warming is at a rate of approximately 0.3° C./minute.

36. The method of claim 26, wherein the warming is at a rate of approximately 0.1° C./minute.

37. The method of claim 26, wherein the protein is an antibody.

38. The method of claim 37, wherein the antibody is a monoclonal antibody.

39. The method of claim 26, wherein the protein is a pharmaceutical drug substance.

40. The method of claim 26, wherein the method is a process intermediate.

41. A composition comprising a biologically effective amount of the protein in the liquid formulation prepared by the method of claim 26.

42. A method for processing a liquid formulation, the method comprising the steps of:
(1) providing a liquid formulation comprising a protein and a combination of mannitol and sucrose;
(2) gradually freezing the liquid formulation;
(3) gradually thawing the liquid formulation; and
wherein the presence of sucrose suppresses mannitol-induced protein aggregation.

43. The method of claim 42, wherein the method further comprises a step of maintaining the liquid formulation at a frozen state for a period of time following step (2).

44. The method of claim 43, wherein the period of time is 6 months or longer.

45. The method of claim 42, wherein the mannitol is in a concentration no greater than approximately 1 M and the sucrose is in a concentration no greater than approximately 5 M in the liquid formulation.

46. The method of claim 45, wherein the mannitol is in a concentration no greater than approximately 300 mM and the sucrose is in a concentration no greater than approximately 300 mM in the liquid formulation.

47. The method of claim 46, wherein the combined concentration of mannitol and sucrose is approximately 300 mM in the liquid formulation.

48. The method of claim 42, wherein the mannitol and sucrose are at a molar ratio no greater than approximately 3:1 (mannitol:sucrose).

49. The method of claim 42, wherein the mannitol and sucrose are at a molar ratio no greater than approximately 5:1 (mannitol:sucrose).

50. The method of claim 42, wherein the protein is an antibody.

51. The method of claim 50, wherein the antibody is a monoclonal antibody.

52. The method of claim 42, wherein the protein is a pharmaceutical drug substance.

53. A composition comprising a biologically effective amount of the protein processed in the liquid formulation by the method of claim 42.
